(19) United States
(12) Patent Application Publication
(10) Pub. No.: US 2011/0002883 A1
(43) Pub. Date: Jan. 6, 2011

(54) THERAPEUTIC PREPARATIONS CONTAINING WHARTON'S JELLY

(76) Inventors: Boris M. Petrikovsky, Great Neck, NY (US); Igor F. Agaronin, Annandale, NJ (US)

Correspondence Address: LADAS & PARRY LLP, 26 WEST 61ST STREET, NEW YORK, NY, 10023, US

(21) Appl. No.: 12/845,982

(22) Filed: Jul. 29, 2010

Related U.S. Application Data
(63) Continuation of application No. 11/733,365, filed on Apr. 10, 2007.

(51) Int. Cl. A61K 38/20; A61P 17/00

(52) U.S. Cl. 424/85.2

(57) ABSTRACT

Therapeutic preparations for rejuvenation and anti-aging of human skin incorporates Wharton's Jelly or substantial fractions thereof as an active ingredient.

The present invention relates to therapeutic preparations for the rejuvenation and anti-aging of human skin and more particularly to such preparations using naturally occurring and/or synthetically produced Wharton's Jelly.

### BACKGROUND OF THE INVENTION

The skin provides valuable protection and a barrier against environmental factors, including harmful or polluting substances, ultraviolet and infrared radiation, and temperature and moisture fluctuations. As the skin ages, its ability to perform such functions decreases. Such functions may also be diminished by injury or other stresses.

It is also well recognized that skin aging is manifested by wrinkles, aging spots and other discolorations, thinning of the epithelium and weakening and stretching of the muscular support especially on the face, breast, and hands. Various external and internal factors may hasten this process, most common of which is exposure to sun, dust, extreme temperatures as well as certain diseases that affect blood supply to the skin, e.g., diabetes or lupus. Rapid weight loss due to dieting or disease causes skin to sag and lose its underlying support. Exposure to industrial chemicals, solvents, paints, and certain soaps damages skin surface and contributes to aging. Aging skin not only causes distress because of its appearance but can also contribute to infections and wounds due to the partial loss of protective abilities.

A variety of external preparations have been developed in attempts to combat skin aging and to maintain the skin in a healthy condition. Many of such formulations provide only a transient effect, and often provide no meaningful change to the actual dermatological conditions of the skin. Such ingredients commonly include glycerin, sorbitol, and propylene glycol. Other preparations are asserted to have an anti-aging effect include vitamin A, soybean extract, and/seaweed extracts.

Many of the compounds in use are of limited, if any, effect, and may not be suitable for use by all users, causing allergic reactions and/or being incompatible with certain skin conditions. Thus, it can be stated that there are currently no natural effective remedies for skin aging short of recommended precautions, e.g., sunscreens, wearing protective gloves to decrease chemical exposure and special exercises to strengthen facial muscles underneath the skin. Surgical cosmetic operations are widely used to improve the appearance of aging skin, most commonly face-lifts. These procedures are invasive, expensive, and results in short-lived effect. They require anesthesia and may cause various complications including death. They are far from a solution of choice.

It is according a purpose of the present invention to provide skin anti-aging and rejuvenating compositions that are of natural origin, widely compatible with a variety of skin types and conditions, and do not exhibit adverse effects.

Another object of the present invention is to provide a substance (active agent) capable of improving skin elasticity, flexibility, and plasticity.

Yet a further object of the present inventions is to provide a composition to protect skin from changing color.

Another object of the present invention is to provide a therapeutic preparation that has long lasting non-surgical rejuvenative effects on the skin.

A still further object of the present invention is to provide topical therapeutic preparations in a variety of forms such as lotions, creams, ointment, gels, lip pomades, hair lotions, and the like, as well as injectable form, having the above stated objects.

### SUMMARY OF THE INVENTION

The foregoing and other objects and purposes are satisfied by way of the present invention, which is a composition that comprises as an active component Wharton's Jelly or substantial constituents thereof. A preparation including Wharton's Jelly is compounded as a topical or injectable preparation and applied to the area to be treated.

Accordingly, the primary advantage of the present invention is that it provides various topical or injectable preparations for the rejuvenation of the human skin. Another advantage of the present invention is to provide a physiologically active agent or component in the form of Wharton's Jelly capable of improving skin appearance, elasticity, and plasticity. Another advantage of the present invention is to provide a viable alternative to cosmetic and plastic surgical procedures to improve the appearance of aged skin (face lifts, peeling, etc.) by improving skin appearance with Wharton's Jelly-derived cosmetic preparations.

### DETAILED DESCRIPTION OF THE INVENTION

Wharton's Jelly is a gelatinous substance found within the umbilical cord as a mixture of water, gelatin, lipids, proteins, and enzymes. Wharton's Jelly is a rich source of stem cells, fetal specific proteins, fatty acids, and phospholipids, among other components, and also provides some measures of protection to the blood vessels in the umbilical cord. It is named for the English physician and anatomist Thomas Wharton (1614-1673) who first described it in his publication Adenographia, or “The Description of the Glands of the Entire Body”, first published in 1656.

In general, dry Whartons's Jelly is a composition of acid muco polysaccharides (35%), gelatin (25%), hyalaronan (15%), fetal-specific proteins and enzymes, (gelatinase A—metalloproteinase (MMP)-2, 72 KD and gelatinawe B (MMP-9, 92 KD). It also contains a small amount of lipids (phospholipids and glycolipids).

More specifically, Wharton's Jelly is a complex mixture of components. It may be characterized as having a base of proteoglycans supporting a variety of other components. In connection with the present invention, these additional components may be classified as follows:

A. Cytokines
Interleukin 6
Interleukin 1
Interleukin receptor antagonist 1
Interleukin 3
Interleukin 2
Interleukin 4
Interleukin 11
Interleukin 5

B. Chemokines
Interleukin 8

C. Growth factors
Fibroblast growth factor (FGF) (all forms, including base and acid)
Insulin-like growth factor (IGF-I)
Thrombocytic growth factor
Transformative growth factors (alpha and beta)
TGF-beta 3
Epidermal growth factor
Endothelial growth factor
PDGF

D. Adhesion molecules
ICAM-1
VCAM-1
Endothelial E-selectin, and similar

E. Collagen

F. Glycosaminoglycans (GAGs)

G. Gelatinolytic enzymes (degraded collagen)
Gelatinase (MMP-2),
Gelatinase (metalloproteinase (MMP) 2, 72 kD)
Gelatinase B (MMP 9, 92 kD)

H. Cathepsin D

I. Hyaluronic acids

J. Leptin

K. Metalloproteinase

As a skin treatment as contemplated by the present invention, Wharton's Jelly may be used and applied in a pure form, or preferably may be further admixed with an appropriate carrier and/or adjudivant. Alternatively, and particularly if a synthetic form of Wharton's Jelly is compounded from the components set forth above, a blend of its components, as follows, may be administered:
Interleukin 6
Interleukin 1
Interleukin receptor antagonist 1
Interleukin 8
Fibroblast growth factor (FGF) (all forms, including base and acid)
Insulin-like growth factor (IGF-I)
TGF-beta 3
Endothelial growth factor
ICAM-1
VCAM-1
Collagen
Gelatinase
Gelatinase B
Hyaluronic acids.

As known in the art, a wide range of suitable carriers can be employed, including gels, lotions, powders, serums, creams, waxes, gums and the like. Other components that may be incorporated into a formulation having Wharton's Jelly as an active ingredient include absorbents, anti-caking agents, essential oils, anti-foaming agents, bulking agents and colorants, dyes, flavors or fragrances, brighteners, humectants, moisturizers, and the like. In addition to serving as a stand-alone treatment product, a formulation of the present invention may be used as an active ingredient in conjunction with other active agents, compounded into skin-care products, toiletries, and the like, in which case it may be compounded with, for example, anti-viral agents, anti-wrinkle agents, active sunscreen agents, topical anesthetics, anti-oxidants, pH adjusters, anti-acne compositions, sequestering agents, and vitamin compounds, among others.

A cream composition including Wharton's Jelly may be formulated as follows:

| Trade Name | INCI Name | Source/Vendor | % |
|---|---|---|---|
| Deionized Water | Water | N/A | 58.50% |
| Gransil RPS Gel | Cyclopentasiloxane, Polysilicone 11 | Grant | 6.00% |
| Glycerin 99.5% USP | Glycerin | Protameen | 6.00% |
| Aerosil 200 | Silica | Degussa | 5.00% |
| Cetyl Alcohol NF | Cetyl Alcohol | Protameen | 4.00% |
| Gynostemma | Gynostemma Pentaphyllum Extract | BC Research/Grant | 3.50% |
| Biotransformed | | | |
| Florasun-90 | Helianthus Annuus (Sunflower) | Essential Ingredients | 3.00% |
| | Seed Oil | | |
| Wharton's Jelly | | | 3.00% |
| Haloxyl | Hydroxysuccinimide, Chrysin, | Selderma/Croda | 3.00% |
| | Palmitoyl Oligopeptide, Water, | | |
| | Glycerin, Steareth-20 | | |
| Stearic Acid TP132 | Stearic Acid | Protameen | 3.00% |
| Cera Bellina Wax | Polyglyceryl-3 Beeswax | Koster Keunen | 3.00% |
| Triethanolamine 99% | TEA | Dow | 1.00% |
| Phenonip | Phenoxyethanol, | Clariant | 1.00% |
| | Methylparaben, Ethylparaben, | | |
| | Propylparaben, Butylparaben, | | |
| | Isobutylparaben | | |
| | | Total | 100.00% |

The cream is compounded in a conventional manner, such as by the following steps: The deionized water is heated to 80-85 C and the glycerin and triethanolamine are added with stirring. Areosil 200 is then added and mixed. The steric acid, cera bellina wax, cetyl alcohol and Florasun-90 are separately combined at a temperature of 80-85 C and then mixed with the base blend. Each of the Gransil RPS, Gynostemma Biotransformed, Haloxyl and Phenonip is in turn added with sweep mixing. Eyeliss™ at a 3% weight percentage may also be added. Each addition is mixed until uniform before the next component is added. After full blending the Wharton's Jelly is added.

In an analogous manner a gel formulation may be compounded, with the following components:

| Trade Name | PT Code | INCI Name | Source/Vendor | % |
|---|---|---|---|---|
| water | | water | House | 67.98% |
| Natrasol HR 250 | | Hydroxyethylcellulose | Aqualon | 0.70% |
| Hampene | | Tetrasodium EDTA | Jos. Lowenstein | 0.10% |
| Sodium Citrate | | Sodium Citrate | Ruger | 0.22% |
| Sodium Metabisulfate | | Sodium Metabisulfate | Ruger | 0.25% |
| Eukyl K-700 | | Phenoxyethanol, | | |
| | | Benzyl Alcohol, | | |
| | | Tocopherol | Phoenix | 1.00% |
| Citric Acid USP | | Citric Acid | Ruger | 0.10% |
| Polyolprepolymer 15 | | PEG-8/SDMI | | |
| | | Copolymer | Barnet | 0.80% |
| Keltrol CG M | | Xanthan Gum | C.P. Kelco | 0.30% |
| Wharton Jelly | | | | 10.00% |
| Butylene Gycol | | Butylene Glycol | Celanenese | 0.38% |
| Licorice Extract PTH | | Clycyrrhizia Glabra | | |
| | | (Licorice) root extract | Barnet | 0.015% |
| Lecinol S-10 | | Arginine | Ajinomoto | 0.20% |
| Rice Bran BT | | Oryza Sativa (Rice) | | |
| | | Bran Extract | BC Research | 1.00% |
| Sorbic Acid | | Sorbic Acid | | 0.50% |
| Caresse 0428478 | | Fragrance | Expressions Parfumees | 0.35% |
| Cremophor RH40 | | PEG-40 Hydrigenated | | |
| | | Castor Oil | BASF | 1.00% |
| SDA 40B 200 prf | | SD Alcohol 40B | UPC | 15.00% |
| | | | Total: | 100.00% |

In general, the quantity of Wharton's Jelly, expressed as a weight percentage of the formulation, may range from 0.01 to 20%.

The formulation is applied to the body as either a one-time or continuous treatment.

1. (canceled)

2. A skin treatment formulation comprising a physiologically active combination of at least the following as physiologically active ingredients: interleukin-6; interleukin-1; interleukin receptor antagonist-1; interleukin-8; fibroblast growth factor (FGF); insulin-like growth factor (IGF-I); TGF-beta 3; endothelial growth factor; ICAM-1; VCAM-1; collagen; gelatinase; gelatinase B; and hyaluronic acids.

3. A method to combat skin aging, comprising the application of a topical formulation to the skin having an physiologically effective amount of Wharton's Jelly as a physiologically active ingredient.

4. A method to combat skin aging, comprising the application of a topical formulation to the skin having a physiologically effective amount of a combination of interleukin-6; interleukin-1; interleukin receptor antagonist-1; interleukin-8; fibroblast growth factor (FGF); insulin-like growth factor (IGF-I); TGF-beta 3; endothelial growth factor; ICAM-1; VCAM-1; collagen; gelatinase; gelatinase B; and hyaluronic acids as physiologically active ingredients, present in a ratio substantially equal to that as found in Wharton's Jelly.
